(19) United States
(12) Patent Application Publication
(10) Pub. No.: US 2003/0175991 A1
(43) Pub. Date: Sep. 18, 2003

(54) Optically-assisted high precision pregnancy progress monitoring

(76) Inventors: Winston Z. Ho, Hacienda Heights, CA (US); Bo Y. Suh, Los Angeles, CA (US); Fu-Nan Wang, Hacienda Heights, CA (US)

Correspondence Address: Winston Z. Ho, 14541 Langhill Drive, Hacienda Heights, CA, 91745, US

(21) Appl. No.: 10/100,755

(22) Filed: Mar. 18, 2002

(51) Int. Cl. G01N 33/53; G01N 33/558

(52) U.S. Cl. 436/514

(57) ABSTRACT

The disclosure describes how to use an optical-based lateral flow matrix method to accurately monitor and quantify human chorionic gonadotropin (hCG) concentration, 0-150,000 mIU/ml, in specimen from a female. The method relies on using a capture zone immobilized with anti-hCG probes having the capability to capture a maximum of 150,000 mIU/ml of hCG in specimen. The method employs an optical beam to illuminate the entire said capture zone, and precisely quantify said hCG concentration in said specimen by measuring the change of the optical property occurred at said capture zone. The method further includes a mechanism to provide an alarm to inform users according to the test results. According to the invention, the device provides the method for easily and accurately monitoring the daily progress of the pregnancy.

### FIELD OF THE INVENTION

The invention is related to lateral flow matrix devices that utilize optical detection mechanism to quantify human chorionic gonadotropin (hCG), 0-150,000 mIU/ml, with high precision. Apparatus and methods thereof are disclosed for easily and rapidly analyzing hCG samples from female for pregnancy monitoring.

### BACKGROUND OF THE INVENTION

Human chorionic gonadotropin (hCG) is a glycoprotein containing a protein core with branched carbohydrate side chains that usually terminate with sialic acid. The hormone is a heterodimer composed of two nonidentical, noncovalently bound glycoprotein subunits, alpha (α) and beta (β). When the dimmer is dissociated, the hormone activity is lost. There is a single gene for the α-subunit of all four glycoptotein hormones (TSH, LH, FSH, and hCG) located on chromosome. From a physiological point of view, hCG has an important role in maintaining the function of the corpus luteum during the first weeks of pregnancy. No specific receptor is known; it binds to and activated the LH receptor in cells of the corpus luteum in the maternal ovary. The glycation of the subunit has a dominant role in signal transduction, an increase in intracellular cyclic adenosine monophosphate (cyclic AMP). This cyclic AMP increase stimulates the production of progesterone, which prevents menses and thus maintains the pregnancy.

The most important aspect of pregnancy management is an early detection of pregnancy and establishing a diagnosis of viable or non-viable pregnancy as pregnancy in human females can be terminated by unfortunate circumstance, such as spontaneous abortion and ectopic pregnancy. Qualitative tests for hCG in blood or urine are primarily used for the confirmation of pregnancy. Urine hCG tests that is a qualitative measurement of hCG is usually suffice enough to diagnose pregnancy when it has progressed beyond the first week after the first missed period. However, quantitative tests have advantages for prognosis of early pregnancy. Serial determinations of hCG concentration can differentiate a normal pregnancy from an abnormal pregnancy. Further, the downward trends of hCG concentration can prove the prognosis of hCG-producing tumors after surgery. When a pregnancy takes place outside of the uterus, it is called as ectopic pregnancy. Serial quantitative measurements of hCG along with ultrasound is a vital tool, not only important to identify the ectopic pregnancy but also essential to follow the ectopic pregnancy because a negligent follow up of hCG can turn out to be fatal. Therefore, only an attainment of correct diagnosis can render a proper treatment prior its rupture, which doomed to cause a life-threatening hemorrhage. Other areas equally beneficial by obtaining quantitative measurements of hCG are a follow up of abnormal pregnancy and hCG producing tumors after surgery. An abnormal pregnancy can be handled sooner in an adequate manner before the patient being devastated psychologically, if the obtained hCG result could differentiate from that of a normal pregnancy. Those patients with hCG producing tumors who had surgery need to follow with quantitative measurements of hCG periodically. Because the persistence or recurrent hCG producing tumors after surgery can be determined only by a serial quantitative measurement of hCG, that fails to decrease to close to a normal level or plateau is a suggestive of existing disease.

Home test kits that are designed to detect hCG in the urine qualitatively are the most commonly used pregnancy tests. With easy availability on over the counter, these tests are simple enough for the lay people to run the test privately. Most kits provide a single test and use an immunochromatographic method. Immunochromatography, described in U.S. Pat. No. 5,096,837 and No. 5,266,497, are also simple for uses. However, these assays detect only the presence or absence of an analyte quantitatively above a defined cutoff level of the test performed, yet not providing a quantitative measurement of an analyte. U.S. Pat. No. 5,145,789 provides two different figures (+) and (−), which can be visually detected according to the positive and negative analysis. A semi-quantitative method, U.S. Pat. No. 5,786,220, with five capture lines, in contrast to one capture line in the conventional strip, is designed to detect 25, 500, 1,000, 2,000, and 2,500 mIU/mL, respectively. The first line will show color at a threshold concentration of 25 mIU/ml or above, and a second line downstream from the first line is provided for indicate concentration. Although the method is simple to use, it only provide only a discrete, confined, fixed measurement in concentration. U.S. Pat. No. 5,753,517 described a quantitative thrombospondin binding assays based on taking the ratio of analyte of interest in the detection zone to the amount of internal control particles in the control zone. The internal control particles bind to the control reagent immobilized at the control zone. Two antibody-coated particles system often creates unnecessary variations and complexion in the immunochemistry. The method requires two optical systems or one optical system with scanning mechanism to measure the intensity at two different zones.

High precision quantitative immunoassays are commonly performed with very large modular cluster systems or analyzers in the hospitals or commercially available clinical laboratories. Although the modular cluster systems, that can run the test of a large number of samples daily during the week, with robotic liquid handlers have the capability of running 200-800 tests per hour, tests can be run in a batch mode after a large collection of sample for an economical reasons. Consequently, it takes 2-4 days (or longer with involvement of weekend) to obtain a regular test results and more than a half day for the stat results in case of early in the morning. Moreover, these systems are too large to use in near-patient-site tests or in doctor's offices. Further, a large laboratory system is too expensive to equip in practitioner's office ($100,000-$250,000). Therefore, an economical device, yet to be available, with a high precision that has capability of measurements of hCG quantitatively along with a short turn around time of the test results (<10 min.) is essentially important and justified that can be used in doctor's office, for home use, or in emergency room in the hospitals, as the delay of the test results can easily put the patient in jeopardy.

### SUMMARY OF THE INVENTION

The invention is to integrate an optical assay protocol into a lateral flow matrix and apparatus. The membrane is immobilized with anti-hCG probes in the capture zone having the capability to capture 150,000 mIU/ml of hCG in specimen. Because of large amount of probes are utilized in the system, the present invention can measure the whole dynamic range.

It is an object of the present invention to provide optically-assisted quantitative method to measure hCG in biological fluids with high precision (<1 mIU/ml). The illuminating area of the optical beam matched with the capture zone. By this method, the exact concentration of hCG can be measured with high accuracy.

One of the objects of the present invention is to utilize optical system to rapidly (<10 minutes) and easily measure the immunochemistry occurs in the lateral flow system.

It is also an object of the present invention to provide an alarm to inform users according to the test results. It is possible to monitor the daily progress of the the normal and abnormal pregnancy with high confidence level.

The present invention has the advantage of quantitative bioassay, and providing accurate and reproducible results. It should be understood, however, that the detail description and specific examples, while indicating preferred embodiments of the present invention, are given by way of illustration and not of limitation. Further, as is will become apparent to those skilled in the area, the teaching of the present invention can be applied to devices for measuring the concentration of a variety analytes, such as TSH, LH, FSH, progesterone, and etc., in a variety of body fluidic samples.

### BRIEF DESCRIPTION OF THE DRAWING

FIG. 1 is a perspective view of optically-assisted lateral flow matrix.

FIG. 2 is a perspective view of optically-assisted high precision pregnancy assay system with a light source and detector coupled with optical fibers for both light illumination, collection, and detection.

### DETAILED DESCRIPTION OF EMBODIMENTS OF THE INVENTION

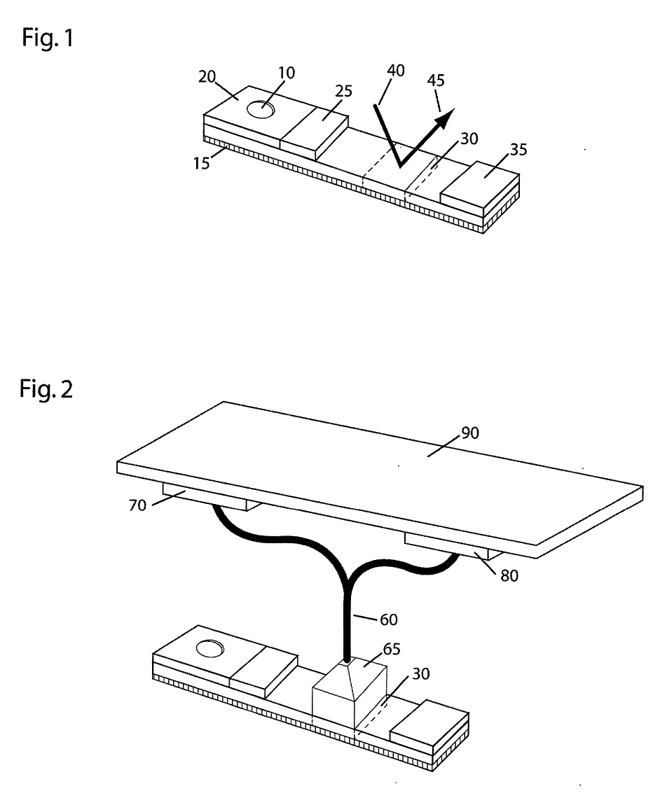

The invention is hereinafter described primarily an optically-assisted lateral flow matrix consisting of a nitrocellulose membrane 15, an application pad 20 at application zone 10, a conjugate release pad 25 at conjugate release zone, a capture zone 30, and an absorbent pad 35 (FIG. 1). The application pad, which is made of membrane, is placed over the lateral flow membrane. The hCG and hormones, and other compounds from the groups consisting of blood, plasma, serum, urine and saliva are transferred across the lateral flow membrane. The conjugate release pad contains label substrates, such as antibody color or dye latex particles, that are specific to the target analyte. At the capture zone, anti-hCG probes are immobilized within a distinct area directly on the membrane. Both label substrate and anti-hCG probes have the capability to bind up to 150,000 mIU/ml of hCG in specimen. An optical system with an optical beam 40 is used to illuminate the capture zone and quantify hCG concentration in specimen by measuring the change of the optical property occurred at the capture zone.

The lateral flow membranes are used to transport the fluids, impregnate the label substrate, and binding the probes. It has a significant effect upon the protein binding observed in a rapid assay, with three key factors affecting performance: pore size, post treatment, and membrane type.

Pore size

Pore size generally has a direct relationship with the wicking rate and the protein-binding of a membrane. In the absence of any special treatment, membranes with larger pore sizes generally have a faster wicking rate but a lower surface area, which results in lower protein binding. Pore size measurement differs between membrane manufacturers, so this attribute should only be used to compare products within a single vendor's suite.

Post treatment

Membrane post treatments may include the addition of surfactants, or a post-washing step to remove the inevitable dust that is present on the membrane surface after manufacture. Treatments are unique to individual manufacturers and it should be established whether these have introduced any materials that may have an effect on protein binding, wicking or membrane aging.

Membrane type

Some membranes have been specifically developed for particular applications. Hydrophobic and hydrophilic properties of the membrane can significantly affect the protein binding and fluid kinetics.

Label latex particles are available in numerous surface chemistry and colors to suit specific needs. To create a color microsphere, one must either covalently attach specific dyes to functional groups on the microsphere surface, or absorb dyes with special solubility properties into the microsphere interior. The advantage of absorbing dyes into the interior is that this allows us to create brighter beads, which are better protected from photolytic degradation and demonstrate longer shelf lives. Protein or other ligands can be physically adsorbed or chemically bound to particles by standard technique. The common colors of the particles are blue, green, and red.

The optical system is designed to ensure the system's compactness, robustness, and reliability. All system components are integrated with solid connections. The optical system (FIG. 2) includes the following parts: LED and compact diode lasers are the light sources of choice for illumination. The technology of LED has improved significantly, high power LED light from Blue (430 nm, 476 nm) to green (530 nm) to red (610 nm, 635 nm) is available. The newly developed LEDs are inexpensive (<$2.0), have very stable output and very long lifetime. The availability of the newly developed laser diode opens up the opportunity for inexpensive optical studies. Red diode laser (638 nm, 1-10 mW) costs $10-$30. The wavelength selection of the light source depends on the color of the particles. With close contact or optical fiber probe illumination, sufficient energy can be provided for illumination. Optical fiber is designed for light delivery. As shown in FIG. 2, the light source 70 can be coupled with optical fibers 60 for both light illumination and signal collection. The light source and detector 80 are assembled on an electrical circuit board 90. The three separate light sources (R, G, B) and a detector are seated on the same circuit board. Depends on which light source is activated, the reflected optical signal 45 is collected through a bifurcated optical fiber probe into the detector. The detector, such as photodiode, measures the reflective light from the capture zone. The photodiode has sufficient efficiency to detect the reflective light. The analog signal will be fed into the analog-to-digital converter, and then to the microprocessor for signal processing, calibration, and quantification.

When sample is deposited to the application zone, the sample containing target analyte is filtered through the application pad. The antigen (Ag), hCG, migrates into the membrane, binds to labeled secondary antibody (Ab*) released from the conjugate release zone, and forms Ag-Ab*. The Ag-Ab* is then captured by the anti-hCG and forms (Ab-Ag-Ab*) complex at the capture zone. Unreacted material is transferred to the absorbent pad at the end of strip. An optical beam is used to illuminate the analyte complex at the capture zone. The reflective signal is collected back to the detector. Various internal control mechanisms can be used for membrane variability and light source fluctuation calibration. The detector measures the reflective signals before (blank) and after analyte appears at the capture zone. Since the reflective light from the blank sample should always return a consistent amount of light, any variation in the measurement should be reflected in the variations of the membrane and light source. More hCG in the sample yields more analyte complex; the reflective signal or ratio (analyte signal/blank signal) reduces with increasing hCG concentration.

According to the invention, the method further includes a mechanism to provide an alarm to inform users according to the test results. For example, different patterns of alarming sounds are generated to inform the user the testing results. It is critical to give a warning and indicate the possibility of abnormal pregnancy symptom. The method extends the pregnancy test to provide accurately monitoring daily pregnancy progress.

The claim of the invention is:

1. An optical-based method for accurately monitoring and quantifying hCG concentration in specimen from a female, said method comprising the steps of:
(a) applying said specimen to a lateral flow matrix, wherein said specimen containing said hCG and said specimen is selected from the groups consisting of blood, plasma, serum, urine and saliva; said lateral flow matrix comprising sample application zone for introducing said specimen, conjugate release zone impregnated with label substrates, and capture zone immobilized with anti-hCG probes; said conjugate lease zone located between said sample application zone and said capture zone;
(b) transferring said specimen and said label substrate to said capture zone through said lateral flow, said hCG and said label substrates binding to said anti-hCG probes and forming (anti-hCG)-(hCG)-(label substrate) complex in said capture zone; and
(c) illuminating said capture zone with an optical beam, and quantifying said hCG concentration in said specimen by measuring the change of the optical property occurred at said capture zone.

2. The method as defined in claim 1, wherein said anti-hCG probes in said capture zone has a capability to capture 150,000 mIU/ml of said hCG in said specimen.

3. The method as defined in claim 1 or 2, wherein said optical method includes a mechanism providing an alarm to inform users according to the test results.

4. The method as defined in claim 1 or 2, wherein said optical beam illuminates the entire said capture zone.

5. The method as defined in claim 1 or 2, wherein said label substrates are color-coated particles.

6. The method as defined in claim 5, wherein said color-coated particles are labeled with anti-hCG.

7. The method as defined in claim 1, wherein said optical beam having wavelength between 350 nm-750 nm.
